(19) United States
(12) Patent Application Publication
(10) Pub. No.: US 2015/0164087 A1
(43) Pub. Date: Jun. 18, 2015

(54) Bacillus Subtilis Strain

(71) Applicant: Novozymes A/S, Bagsvaerd (DK)

(72) Inventor: Yaowei Kang, Blacksburg, VA (US)

(21) Appl. No.: 14/630,002

(22) Filed: Feb. 24, 2015

Related U.S. Application Data
(62) Division of application No. 12/490,090, filed on Jun. 23, 2009.
(60) Provisional application No. 61/074,909, filed on Jun. 23, 2008.
(60) Provisional application No. 61/078,813, filed on Jul. 8, 2008.

(51) Int. Cl. A01N 63/00; C12N 1/20; C11D 3/38; A61L 9/01

(52) U.S. Cl. CPC A01N 63/00; A61L 9/01; C12N 1/20; C11D 3/381

(57) ABSTRACT

The present disclosure relates to the use of an aqueous mixture of an odor neutralizer component, an enhancer component for microbial activity, and a microbial component. This composition and methods of use are designed to provide short- and long-term odor control effects and is environmentally friendly and economical for use.

### CROSS-REFERENCE TO RELATED APPLICATIONS

This application is a divisional of U.S. application Ser. No. 12/490,090 filed on Jun. 23, 2009 which claims the benefit under 35 U.S.C. 119 of U.S. provisional application Nos. 61/074,909 and 61/078,813 filed Jun. 23, 2008 and Jul. 8, 2008, respectively, the contents of which are fully incorporated herein by reference.

### CROSS-REFERENCE TO DEPOSITED MICROORGANISMS

The present application refers to deposited microorganisms. The contents of the deposited microorganisms are fully incorporated herein by reference.

### FIELD OF THE INVENTION

The present invention relates to Bacillus subtilis strain NRRL B-50147, compositions comprising the Bacillus subtilis strain, and deodorizing liquid compositions which are designed to be applied in the areas of pet care, toilet care, carpet care, and garbage collections or processes, management of industrial wastes, including sludge processing, landfill and composting, and odor control of livestock production processes and other organic wastes.

### BACKGROUND OF THE INVENTION

Offensive odors are generated from various sources, including pet wastes, toilets, carpets, garbage collections and processes, animal manure, industrial waste sites such as sludge processes, landfill sites, and composting sites, etc. Among the odorous compounds, amines, ammonia, hydrogen sulfide, organic acids, and mercaptans are very often found in the malodors from various sources and they are, respectively, the products of decomposition and other reactions of organics and nitrogen-and sulfur-containing materials. Offensive odors have posed a series of social and environmental problems including hazards to mental health, damages to health of humans, especially the workers in odor-generating facilities, and negative effects on animal growth and reproduction.

Conventionally, masking agents, such as fragrances, have been used to cover up an objectionable odor with a more desirable one. However, masking agents may not actually reduce concentrations of odorous gases and they also quickly lose their effectiveness due to vaporization and microbial break down. Chemical oxidizing agents and germicides have also been used to control odors by altering or eliminating bacterial action responsible for odor production. These chemical agents, however, will destroy the beneficial microbial activity in the treated systems.

Furthermore, some of them might not be safe for humans and animals and are usually expensive for use. Other deodorizing approaches include use of adsorbents, neutralizers, and biological degradation or conversion. Adsorbents are products with a large surface area that may be used to adsorb the odors before they are released to the environment. Neutralizers are materials which react with odorous compounds to form odorless ones. Biological degradation or conversion can eliminate odors through biochemical digestive processes. The biological approaches include: 1) use of externally added microbes and enzymes; and 2) use of enhancing agents to ensure or increase the activity of added microbes and indigenous microbial populations.

Use of a biological approach is a promising one, since it can eliminate odors through biodegrading odor sources including organics and nitrogen- and sulfur-containing materials, thus providing long-term odor control. This approach is environmentally friendly and usually economical. Because odorous compounds are very volatile, rapid containment of odors, through using adsorbents and/or neutralizers, is usually necessary before the odors are released to the environment.

U.S. Pat. No. 4,879,238 (Hata) discloses the deodorization by using a single strain or a few strains of bacteria.

Further, U.S. Pat. No. 4,996,055 (Kurasawa) discloses a deodorant that contains butyric acid bacteria and Bacillus subtilis as effective components for treating excrement of various animals and other sources of foul odors.

Bacillus subtilis strain SB3106 is included in products sold by Novozymes for cleaning and odor control applications in which enzymes help remove organic soils that cause inorganic soils to cling and promote malodors and for drainline/grease trap applications in which the strain helps degrade grease and organics that cause drainline build-ups that cause blockages.

It is an object of the present invention to provide an environmentally favorable and effective biological agent for a broad range of applications such as deodorizing liquid compositions.

### SUMMARY OF THE INVENTION

The present invention relates to a biologically pure culture of Bacillus subtilis strain NRRL B-50147. Bacillus subtilis strain NRRL B-50147 is a bacteriophage-resistant (phage-resistant) variant of Bacillus subtilis strain SB3106. In order to propagate Bacillus subtilis strain NRRL B-50147 to a number large enough to allow broad application of this strain, repeated, large-scale fermentation is required. It is known that the natural introduction of native bacteriophage can occur in standard large-scale fermentation systems over repeated growth events or batches. Such an infection can rapidly lead to a complete loss of the culture within hours or days, negating the ability to provide the strain for practical applications. Bacillus subtilis strain NRRL B-50147 is resistant to such a phage, and therefore maintains growth and realizes the benefits described herein.

Bacillus subtilis strain NRRL B-50147 is able to produce amylase, which catalyze the degradation of the principal chemical components of drain residues, such as starches.

This invention also relates to a liquid deodorizing composition comprising Bacillus subtilis strain NRRL B-50147 in an aqueous solution, e.g., distilled water, tap water, a saline solution or other aqueous solution.

The present invention is also directed to a liquid formulation which enhances plant root development.

The present invention is also directed to a drain opener formulation comprising Bacillus subtilis strain NRRL B-50147.

The present invention also relates to a sanitizing composition comprising Bacillus subtilis strain NRRL B-50147 in an aqueous solution.

### DETAILED DESCRIPTION OF THE INVENTION

### Culture

The present invention is directed to a biologically pure culture of Bacillus subtilis strain NRRL B-50147.

### Liquid Deodorant Compositions

The present invention is also directed to a composition comprising Bacillus subtilis strain NRRL B-50147 in an aqueous solution. This composition is designed to provide short-and long-term odor control effects and is environmentally friendly and economical for use.

An operable concentration range for Bacillus subtilis strain NRRL B-50147 is from about 1×10⁵ to 1×10¹⁰ CFU/ml, e.g., from about 1×10⁶ to 1×10⁸ CFU/ml, with a preferred concentration being about 1×10⁸ CFU/ml, such as about 1×10⁷ CFU/ml of the formulation.

### Odor Neutralizer Components

The deodorant compositions of the present invention may further comprise an odor neutralizer, which is an agent that can rapidly interact, by chemical reactions, with odorous compounds to produce odorless compounds. These agents should not rely on the masking mechanism of a perfume to control odors. In addition, these agents must be safe for use and cost effective. Neutralizers must be compatible with the mircrobial components.

In one embodiment of the present invention, the neutralizer is propylene carbonate, which has the molecular formula C₄H₆O₃. A preferred product of propylene carbonate is available from commercial vendors such as Huntsman Chemical Corporation.

In combination with other components of the composition, propylene carbonate can effectively reduce odors, including amine and ammonia odors such as trimethylamine, dimethylamine, and ammonia, which are the major target odorous compounds. In addition, propylene carbonate does not inactivate the microbial components even after a long period of contact.

Other odor neutralizing compounds, such as sodium citrate, sodium bicarbonate, and sodium carbonate, may also be used in the formulation of this invention.

Preferably, the odor neutralizing is present in an amount of 1-15 wt. %, such as 2-10 wt. % of the composition.

### Other Microbial Components

Viable microorganisms, or mixtures thereof, which are capable of growing on and degrading common domestic, industrial, pet, and animal wastes, capable of surviving the formulations, and compatible with the formulations, and do not produce malodor while performing, may be used in the invention.

Other microorganisms which can be used in the compositions of the present invention include strains of Alcaligens, Bacillus, Enterobacter, Klebsiella, Lactobacillus, Nitrobacter, Nitrosomonas, Pseudomonas, and Streptococcus, which are known to produce enzymes which are capable of breaking down organic material which can cause odors on carpets or other fibrous materials.

### Other Ingredients

Other ingredients may be used in the deodorant compositions of the present invention, including surfactants, fragrances, and dyes.

Surfactants can wet and emulsify insoluble waste materials present in the treated system and inclusion of surfactants in the composition of the invention will add to it a cleaning capability. Furthermore, surfactants can be used to break down the insoluble wastes therefore increasing the availability of them to microbial degradation. Suitable surfactants for the invention include nonionic and anionic types. Preferably, the surfactant is present in an amount of 0-8 wt. %, such as 0-6 wt. % of the composition.

Fragrance and dye can be optionally added to mask the odor and to control the color of the composition of the invention, respectively, and for market appeal.

The fragrance and dye must be compatible with other ingredients of the composition.

### Drain Opener Formulations

The present invention is also directed to a drain opener formulation comprising Bacillus subtilis strain NRRL B-50147 in an aqueous medium.

The drain opener formulation may further comprise surfactant(s) and/or preservative(s). The product has numerous advantages over currently available drain openers; such as activity at pH's closer to neutral, and solubilizing ability for soaps, fats, oils and greases. It further provides for biological activity specific to carbohydrates, and establishes a biofilm in the drains and on downstream surfaces to continuously aid the natural biodegradative process.

The composition of the present invention comprises a stable suspension of viable microorganisms, surfactant(s), preservatives, and optional fragrances in an aqueous medium with a preferred pH of approximately 5 to 6.

An operable concentration range for the microorganisms is from about 1×10⁶ to 1×10⁹ CFU/ml, with a preferred concentration being about 1×10⁸ CFU/ml, such as about 1×10⁷ CFU/ml of the formulation.

Unlike typical detergents, which predominately only clean surfaces, the surfactant in the formulation of the present invention can solubilize grease and to make it bioavailable. The surfactant can be any readily biodegradable surfactant, or a mixture of surfactants with low toxicity for the microorganisms contained within the system. The surfactant(s) should have a high grease solubilizing capability. Ionic surfactants or blends of nonionic/ionic surfactants having a hydrophile/lipophile balance approaching 10 are particularly preferred for the necessary grease solubilization. Typical surfactants suitable for use with the present invention include n-alkyl benzene sulfonates and alkyl sulfonates. Preferred nonionic surfactants include aliphatic alcohol alkoxylates, alcohol ethoxylates, polyalkylene oxide copolymers, alkyl phenol alkoxylates, carboxylic acid esters, carboxylic amides, and others. The surfactant is present in a concentration from about 3 to 10 weight percent.

The pH of the solution should be maintained as near as possible to neutral to insure adequate bacterial activity, and to minimize health risk, but be in a range compatible for surfactant activity and conducive to the survival of the bacteria. An operable pH range can be between about 3 to 10.

A preservative such as paraben, methyl paraben, or 1-2-benzisothiazolin-3-one is added to inhibit or prevent the growth of undesirable microbial contaminants in the product. The necessity for a preservative is greatest when the pH is near neutral, and the least when the pH is at the extreme ends of the operable range. The concentration of the preservative is determined by the vendor's recommendations. A typical concentration range for the preservative used in the example is from about 0.075 to 0.75 weight percent.

An additional optional preservative can be added specifically to preserve the spore form of the microorganisms. Methyl anthranilate in concentrations of from about 25 to 50 ppm (w/v) by weight has been found to be a satisfactory additive.

Optionally a chelating agent is added to enhance stabilization of the formulation.

A fragrance can optionally be added to mask the odor of the product components, and for market appeal. The fragrance must be compatible with the other components of the formulation.

### Sanitizer Formulations

The present invention also relates to sanitizer formulations comprising Bacillus subtilis strain NRRL B-50147. The formulations comprise a suspension of a sanitizing composition, bacterial spores, and surfactants all contained in an aqueous solution. These formulations have the advantages of being a good surface cleaning agent and a good sanitizer along with providing the long term effect of beneficial bacteria that control pathogens and degrade wastes both on the surface and in the sewage system receiving the surface rinsate.

Sanitizing agents or composition and disinfectants belong to the same category of antimicrobial (active) ingredient. Antimicrobial (active) ingredients are compounds that kill microorganisms or prevent or inhibit their growth and reproduction and that contribute to the claimed effect of the product in which it is included. More specifically, a sanitizer is an agent that reduces the number of microbial contaminants or pathogens to safe levels as judged by public health requirements.

The surfactant component functions to clean the surface by removing the soil, dirt, dried urine and soap and helps in sanitizing the surface. The sanitizing composition sanitizes the surface (kills pathogens) and preserves the formulation from contamination by unwanted microorganisms. The bacterial spores and vegetative cells function to seed the waste collection system, control odor and provide a healthy dominant microbial population that inhibits the growth of pathogens through substrate competition, production of antibiotics, etc.

In one embodiment of the present invention, the composition comprises 1,2-benzisothiazolin-3-one (Proxel), tetrasodium ethylenediaminetetraacetate (EDTA), and isopropyl alcohol (IPA) at a selected range of concentrations, combined with other components of the formula, can effectively inactivate indicator organisms. This sanitizing composition preferably is at neutral pH and does not contain chlorine-related materials, which are commonly used as sanitizers. Consequently, this sanitizing composition is more environmentally friendly and less or not corrosive.

When the formulation is applied to a bathroom fixture, sink, toilet bowl, etc., it can be sprayed or squeezed out of a container directly onto a surface or brush. The formulation is then left on the surface or scoured against the surface with a brush for not less than 10 minutes. The product is then flushed or rinsed with water and discharged from the fixture.

The formulations of the invention contain sanitizing agents, bacterial spores, and surfactants. Fragrance and dye are also added to control smell and color of the formulations, respectively. Depending on the intended use, the formulation can optionally contain an abrasive. While the key components remain the same, different thickening agents might be used in the formulation with and without an abrasive.

Although many sanitizing agents can be used for inactivating pathogens on surfaces, not all of them can be used in the present invention. This is because the sanitizing agents used in this invention are not only required to inactivate pathogens effectively, but must not have negative effects on the stability and activity of the bacterial spores contained in the formulation. In addition, the sanitizing agents are required to be relatively friendly to the environment, and should not cause skin sensitization, and should not corrode the construction materials of the fixtures on which they are used.

In an embodiment, the sanitizing composition is composed of Proxel, EDTA, and IPA at selected ranges of concentrations. The maximum concentration of Proxel not likely to cause skin sensitization is about 2,900 mg/L. The suitable concentration ranges of Proxel, Versene (Versene contains 39% EDTA), and IPA for producing a 4 log reduction in the count of an indicator organism in 10 minutes are 0.087 to 0.29% (vol.), 0.36 to 1.19% (vol.), and 3.5 to 7% (vol.), respectively. An additional compound, methyl anthranilate, may also be used in the formulations of the invention. The purpose of using methyl anthranilate is to assist in preservation of the formulations.

Other sanitizing agents, such as quaternary ammonium compounds (QACs), nitro-containing organosulfur and sulfur-nitrogen compounds, may also be used in the formulation of this invention.

An operable concentration range for the microorganisms is from 1×10⁵ to 1×10⁹ CFU/ml, such as 10⁷ CFU/ml (CFU, colony forming unit) of the formulation.

### Surfactants

Surfactants are also an essential component in the sanitizer formulations of the present invention. The surfactants can wet and emulsify soil, including dirt, dried urine, soap, etc., present on a dirty surface. In addition, surfactants aid in the sanitization of the surface. Unlike surfactants usually used for surface cleaning, the surfactants used in the present invention have low toxicity for the microorganisms contained within the formulation. A single surfactant or a blend of several surfactants can be used.

Nonionic surfactants are generally preferred for use in the compositions of the present invention since they provide the desired wetting and emulsification actions and do not significantly inhibit spore stability and activity. Nonionic surfactants are surfactants having no electrical charge when dissolved or dispersed in an aqueous medium. Preferred nonionic surfactants include aliphatic alcohol alkoxylates, alcohol ethoxylates, polyalkylene oxide copolymers, alkyl phenol alkoxylates, carboxylic acid esters, carboxylic amides, and others.

Anionic surfactants or mixtures of anionic and nonionic surfactants may also be used in the formulations of the invention. Anionic surfactants are surfactants having a hydrophilic moiety in an anionic or negatively charged state in aqueous solution. Commonly available anionic surfactants include sulfonic acids, sulfuric acid esters, carboxylic acids, and salts thereof.

### Abrasives, Thickening Agents, Fragrance, and Dyes

Abrasives are water-insoluble solid particles. The purpose of using abrasives is to provide deep scouring and cleaning. Depending on the application, abrasives may be optionally used in the formulation of the invention. Suitable abrasives include calcium carbonate, magnesium carbonate, silica, etc. The preferred particle size of the abrasive ranges from about 90 to 325 mesh.

Since the specific gravity of bacterial spores is usually higher than that of water, a thickening agent needs to be used in this invention to suspend the spores. Suitable aqueous thickening agents include: polyacrylic acid, polystyrene, polyvinyl alcohol, polypropylene, etc. A preferred thickening agent for suspending bacterial spores is polyacrylic acid (e.g., Acrysol TT615 from Rohm and Haas Co.). If an abrasive is used in the formulation, thickening agents in addition to polyacrylic acid might be needed to maintain the suspension of the abrasive.

A fragrance and a dye can be optionally added to mask the odor and to control the color of the product components, respectively, and for market appeal. The fragrance and dye must be compatible with the other components of the formulation.

### Deposit of Biological Material

A Bacillus subtilis strain was deposited under the terms of the Budapest Treaty on Jun. 18, 2008 with the Agricultural Research Service Culture Collection, 1815 North University Street, Peoria, Ill. 61604, U.S.A., under accession number NRRL B-50147. The deposit shall be maintained in viable condition at the depository during the entire term of the issued patent and shall be made available to any person or entity for non-commercial use without restriction, but in accordance with the provisions of the law governing the deposit.

The following examples are given as exemplary of the invention but without intending to limit the same.

### EXAMPLES

### Materials & Methods

### Media and Reagents:

Chemicals used as buffers and reagents were commercial products of at least reagent grade.

Plate Count Broth (cat. #275120, Difco-Becton Dickinson, Sparks, Md.) (“PCB”)

Standard Methods agar plates (SMA plates) (Smith River Biologicals, Ferrum, Va. cat. #11-00450)

Marine Agar 2216 (cat. #212185, Difco-Becton Dickinson, Sparks, Md.)

Marine Broth 2216 (cat. #279110, Difco-Becton Dickinson, Sparks, Md.)

Bacto-Peptone (cat. #211677, Difco-Becton Dickinson, Sparks, Md.)

Bacto-Tryptone (cat. #211705, Difco-Becton Dickinson, Sparks, Md.)

Yeast Extract (LD) (cat. #210933, Difco-Becton Dickinson, Sparks, Md.)

Soluble Starch (cat. #S-2630, Sigma, St. Louis, Mo.)

R1 and R2 buffers (cat. #11876473 316; Roche, Indianapolis, Ind.)

### Equipment

Konelab Arena 30 (Thermo Electron Corporation, Vantaa, Finland)

Synergy Kinetic Microtiter Plate Reader (BioTek, Winooski, Vt.)

### Example 1

### Enzyme Production Procedure:

Enzyme production medium is used according to the following recipe: Base Media (all values in g/L unless otherwise noted)

| | |
|---|---|
| Bacto-Peptone | 2.5 |
| Bacto-Tryptone | 2.5 |
| NaCl | 2.5 |
| Yeast Extract | 3 |
| Soluble Starch | 1 |

The components are mixed in DI water and autoclaved for 20 minutes.

10 ml overnight cultures of strains are grown in PCB at 35° C. with shaking at 200 rpm. The next day, 0.2 ml of this culture is used to inoculate 100 ml of enzyme production medium. This culture is grown at 35° C. with shaking at 200 rpm. All culture flasks are grown for 80 hours at 35° C. with shaking at 200 rpm.

Over the course of 80 hours at 8-12 hour frequencies, 3 ml of culture is removed, centrifuged, filtered and 2 ml of the filtrate is added to a plastic tube containing 1.0 ml of sterile 50% glycerol. The tube is labeled and stored at -20° C. until all samples are ready for analysis.

### Amylase Assay:

Alpha-amylases (1,4-α-D-glucanohydrolases, E.C. 3.2.1.1) catalyze the hydrolytic degradation of polymeric carbohydrates such as amylose, amylopectin and glycogen by cleaving 1,4-alpha-glucosidic bonds. In polysaccharides and oligosaccharides, several glycosidic bonds are hydrolyzed simultaneously. Maltotriose, the smallest such unit, is converted into maltose and glucose, albeit very slowly. The kinetic method described here is based on the well-proven cleavage of 4,6-ethylidene-(G7)-1,4-nitrophenyl-(G1)-α,D-maltoheptaoside by alpha-amylase and subsequent hydrolysis of all the degradation products to p-nitrophenol with the aid of alpha-glucosidase. This results in 100% liberation of the chromophore.

This process has been automated in the Konelab Arena 30 with the following steps:

1) 200 microliters of R1 reagent is pipetted into cuvette,

2) 16 microliters of sample is added to cuvette,

3) Mixture is incubated for 300 seconds to obtain temperature of 37° C.,

4) 20 microliters of R2 reagent is pipetted into cuvette and mixture is incubated for 180 seconds, and

5) Absorption is measured every 18 seconds at 405 nm for a total of 7 measurements for each sample.

Defined oligosaccharides are cleaved under the catalytic action of alpha-amylases. The resulting PNP derivatives are cleaved directly to PNP by the action of alpha-glucosidase and the color intensity of the p-nitrophenol formed is directly proportional to the alpha-amylase activity and is measured spectrophotometrically.

5 ethylidene-G₇PNP+H₂O→2 ethylidene-G₅+2 G₂PNP+2 ethylidene-G₄+2 G₃PNP+ethylidene-G₃+G₄PNP (1)

2 G₂PNP+2 G₃PNP+G₄PNP+14H₂O→5 PNP+14G (2)

Reaction (1) is mediated by the amylase added from the standard or sample. Reaction (2) is mediated by the glucosidase provided in the kit.

### Unit Definition

BAN is an alpha-amylase available from Novozymes. The analytical standard was supplied at 360 KNU(B)/g=360 NU(B)/mg.

### Specificity and Sensitivity

Because each amylase will have a different specificity, the samples should be diluted such that the final slopes read from the Konelab are between 0.05 and 0.50 to make sure that the experimental samples fall within the scope of the standard curve.

Bacillus subtilis strain NRRL B-50147 produced amylase activity in these assays.

### Example 2

### Phage Sensitivity Assay

Bacillus subtilis strain NRRL B-50147 and Bacillus subtilis strain SB3106 were grown in buffered plate count broth (BPCB: 17 g m-Plate Count Broth, 20 ml of pH 7 buffer made with 1 part 9.078 g/L KH₂PO₄ and 1.5 parts 9.476 g/L of K₂HPO₄, pH adjusted to 7) to a density of approximately 0.2 absorbance units at 590 nm wavelength. 100 microliters of each culture were delivered to wells of a 96 well BD Oxygen Biosensor microtiter plate (Catalog #353830, BD Lifesciences, San Jose, Calif.). The cultures were diluted in additional BPCB and 0.1 and 0.01× dilutions of the cultures were delivered to additional wells of the same plate. Each dilution of bacterial culture received 100 microliters of five different concentrations of phage challenge as follows: 1× (˜10¹⁰ pfu/ml), 0.1×, 0.01×, 0.001×, and 0.0001×. The diluent for the phage was BPCB. One well of each bacterial culture dilution received 100 microliters of plain BPCB instead of phage and thus served as the control well. Plates were read on a kinetic plate reader (BioTek Synergy, Winooski, Vt.) at 485/20 nm excitation, 645/40 nm emission at 20 minute intervals for 20+ hours with 10 seconds of mixing at level 4 before each read. The BD Oxygen Biosensor microtiter plates contain an oxygen sensitive fluorophore that fluoresces when the cell culture in the well consumes oxygen and thus fluorescence intensity correlates to culture growth rates and general health. Data was analyzed by comparing the fluorescent O₂ consumption curves of Bacillus subtilis strain NRRL B-50147 to the Bacillus subtilis strain SB3106 at the various bacteria and phage ratios. Increasing fluorescence (bacterial growth) without decreases or plateaus (lysis or decreased growth rate) in the presence of phage was interpreted as resistance to phage. Bacillus subtilis strain NRRL B-50147 outperformed Bacillus subtilis strain SB3106 in this way at multiple cell and phage densities examined. At 0.1× and 0.01× cell culture concentration, Bacillus subtilis strain SB3106 succumbed to phage pressure at most phage concentrations tested by showing a severe depression in O₂ consumption which lasted at least 3 hours, whereas Bacillus subtilis strain NRRL B-50147 showed ample and prolonged proliferation at all phage concentrations.

### Example 3

Petri Plate V. harveyi Zone of Inhibition

Bacillus subtilis strain NRRL B-50147 and V. harveyi (ATCC 25919) were grown separately in plate count broth or marine broth, respectively, for 18 to 20 hours at 28° C. with shaking. V. harveyi culture was swabbed to form a lawn on the surface of Marine Agar (Difco) and a 5 mm hole was bored into the agar with a sterile stainless steel tube. 50 microL of Bacillus subtilis strain NRRL B-50147 liquid culture was delivered into the hole in the agar and the plate was incubated for 24 hours at 30° C., agar side down. Inhibited V. harveyi lawn in proximity to the hole was scored as positive biocontrol for Bacillus subtilis strain NRRL B-50147. The diameter of the zone of inhibition (including the hole) was measured in millimeters (mm) to allow semi-quantitative assessment of control. Bacillus subtilis strain NRRL B-50147 zone diameter was 8 mm.

### Example 4

Petri Plate Pseudomonas aeruqinosa Zone of Inhibition

Bacillus subtilis strain NRRL B-50147 and Pseudomonas aeruginosa Pa-01 (gift from Montana State University, lab of Ann Camper) were grown separately in plate count broth for 18 to 20 hours at 28° C. with shaking. Pseudomonas culture was swabbed to form a lawn on the surface of Standard Methods Agar and a 5 mm hole was bored into the agar with a sterile stainless steel tube. 50 microL of Bacillus subtilis strain NRRL B-50147 liquid culture was delivered into the hole in the agar and the plate was incubated for 24 hours at 30° C. and then 3 days at room temperature (˜21° C.), agar side down. Inhibited Pseudomonas lawn in proximity to the hole was scored as positive biocontrol for Bacillus subtilis strain NRRL B-50147. The diameter of the zone of inhibition (including the hole) was measured in millimeters (mm) to allow semi-quantitative assessment of control. Bacillus subtilis strain NRRL B-50147 zone diameter was 14 mm.

While specific embodiments of the invention have been illustrated and described herein, it is realized that modifications and changes will occur to those skilled in the art. It is therefore to be understood that the appended claims are intended to cover all modifications and changes as fall within the true spirit and scope of the invention.

1. A method of deodorizing composition comprising contacting a surface with a liquid deodorizing composition comprising Bacillus subtilis strain NRRL B-50147 in a stable aqueous medium.

2. The method of claim 1, wherein Bacillus subtilis strain NRRL B-50147 is present in a concentration of from about 1×10⁵ to 1×10¹⁰ per ml.

3. The method of claim 1, wherein the liquid deodorizing composition comprises an odor neutralizing component which functions to provide for rapid odor reduction.

4. The method of claim 3, wherein the odor neutralizing component comprises propylene carbonate.

5. The method of claim 3, wherein the odor neutralizing component is at least one selected from the group consisting of sodium citrate, sodium bicarbonate, and sodium carbonate.

6. The method of claim 1, wherein the liquid deodorizing composition comprises one or more microbes selected from the group consisting of Alcaligens, Bacillus, Enterobacter, Klebsiella, Lactobacillus, Nitrobacter, Nitrosomonas, Pseudomonas, and Streptococcus.

7. A method of enhancing plant root development, comprising applying to a plant Bacillus subtilis strain NRRL B-50147.

8. A method of using a drain opener formulation comprising contacting a drain with Bacillus subtilis strain NRRL B-50147 and a surfactant.

9. A method of using a sanitizing composition comprising contacting a surface with Bacillus subtilis strain NRRL B-50147 and a surfactant.

10. An isolated biologically pure culture of Bacillus subtilis strain NRRL B-50147.
